(19) United States
(12) Patent Application Publication
(10) Pub. No.: US 2025/0157667 A1
(43) Pub. Date: May 15, 2025

(54) Predicting Recurrent Urolithiasis And Decision Support Tool

(71) Applicant: Cerner Innovation, Inc., Kansas City, MO (US)

(72) Inventor: Douglas S. McNair, Leawood, KS (US)

(73) Assignee: Cerner Innovation, Inc., Kansas City, MO (US)

(21) Appl. No.: 19/026,005

(22) Filed: Jan. 16, 2025

Related U.S. Application Data
(63) Continuation of application No. 15/947,286, filed on Apr. 6, 2018.
(60) Provisional application No. 62/482,679, filed on Apr. 6, 2017.

(51) Int. Cl. G16H 50/30; G01N 33/493; G01N 33/68; G01N 33/84; G16H 10/40; G16H 50/20

(52) U.S. Cl. CPC G16H 50/30; G01N 33/6893; G16H 50/20; G01N 33/493; G01N 33/84; G01N 2800/345; G16H 10/40

(57) ABSTRACT

Decision support technology is provided for use with patients prone to recurrent urolithiasis. A mechanism is provided to determine a forecast of urolithiasis for a patient over a future time interval. The forecast may be based on temporal patterns in urinalysis parameters of the patient. In one embodiment, the mechanism utilizes a time series Hölder exponent and recurrence quantification analysis (RQA) recurrence rate to generate a forecast of recurrent symptomatic urolithiasis for a future time, such as a multi-year time horizon. Based on the generated forecast, one or more intervening actions may be carried out automatically or may be recommended, including modifying a care program for the patient, automatically scheduling interventions or consultations with specialist caregivers, or generating notifications such as electronic messages or alerts.

### INCORPORATION BY REFERENCE; DISCLAIMER

Each of the following applications are hereby incorporated by reference: application Ser. No. 15/947,286 filed on Apr. 6, 2018; Application No. 62/482,679 filed on Apr. 6, 2017. The applicant hereby rescinds any disclaimer of claims scope in the parent application(s) or the prosecution history thereof and advises the USPTO that the claims in the application may be broader than any claim in the parent application(s).

### BACKGROUND

The lifetime prevalence of symptomatic kidney stones is approximately ten to fifteen percent. During an acute episode of urolithiasis, spontaneous passage occurs in about sixty-eight percent of stones smaller than five millimeters and forty-seven percent between five-to-10 millimeters. Thus, larger stones and many smaller stones require some form of intervention. While shock-wave lithotripsy and endourologic approaches have greatly improved treatment of urolithiasis, monotherapy does not seem to be sufficient for most stones. Up to thirty-five percent of patients with ureteral stones may not be rendered stone-free and thus require multiple interventions. Moreover, despite a major shift towards ureteroscopy and ureteroscopic lithotripsy, the rate of ancillary procedures, repeat encounters, and complication rates have not fallen dramatically.

Pain resulting from stones that obstruct the ureters or other parts of the urinary collecting system is notoriously severe, and is associated with considerable suffering, morbidity, and health care services utilization as well as anticipatory dread experienced by patients. The cumulative stone recurrence rate is approximately fifty percent in ten years and seventy-five percent in twenty years but varies by stone composition/stone formation physiology-chemistry. Although trend data is not entirely reliable, the prevention of recurrences has not improved. For some, such as patients who have polycystic kidney disease (PKD), symptomatic stone recurrence may occur multiple times each year, although recurrences may partly be due to provider variation in evaluation and management.

### SUMMARY

This summary is provided to introduce a selection of concepts in a simplified form that are further described below in the detailed description. This summary is not intended to identify key features or essential features of the claimed subject matter, nor is it intended to be used in isolation as an aid in determining the scope of the claimed subject matter.

Improved screening, monitoring and decision support technology is provided for use with patients prone to recurrent urolithiasis, including determining a likelihood of urolithiasis for a patient over a future time interval. In particular, a mechanism is provided to determine a numerical probability of future recurrent urolithiasis in a patient based on temporal patterns in urinalysis parameters of the patient. In one embodiment, the mechanism utilizes a time series Hölder exponent and recurrence quantification analysis (RQA) recurrence rate to generate a forecast of recurrent symptomatic urolithiasis for a future time, such as a multi-year time horizon. The forecast may include a statistical probability or prediction score. Based on the generated forecast and/or score, one or more actions may be carried out automatically or may be recommended, such as, without limitation, intervening in the patient's care, modifying a care program for treating the patient, automatically scheduling interventions or consultations with specialist caregivers, or generating notifications such as electronic messages, which may include recommendations, information, alerts, or alarms, based on the forecast which may be emitted or otherwise provided to the caregiver and/or to the patient.

Some embodiments of the technologies disclosed herein provide a system and method for continually tracking the clinical and physiologic status of a stone-former patient. In some embodiments, a near-term forecast multi-year risk of symptomatic stone recurrence is generated. The forecast may be periodically plotted and displayed or otherwise utilized to visualize a patient's risk trend during his/her urolithiasis prevention management. In this way, physicians, nurses and clinical researchers are enabled to provide improved, safe, and effective care for each patient, especially those who have a prior history of two or more symptomatic stones. Moreover, recognizing a high risk of stone recurrence far enough in advance of the onset of symptoms can guide computer-performed decision support including rational allocation of resources, intensified monitoring and/or treatments that may achieve reduction of risks of symptomatic urolithiasis, decreasing frequency of episodes and length-of-stay in acute care institutions, financial savings, or other benefits.

### BRIEF DESCRIPTION OF THE DRAWINGS

Embodiments of the present technology disclosed herein are described in detail below with reference to the attached drawing figures, wherein:

FIGS. 1A and 1B depict aspects of an illustrative architecture suitable for practicing an embodiment of the present disclosure;

FIG. 2 depicts a flow diagram showing a method utilized by a decision support tool for generating a forecast of urolithiasis for a patient over a future time interval, in accordance with an embodiment of the present disclosure;

FIGS. 3A and 3B depict RQA recurrence rates for a healthy control and an example actual recurrent stone-former patient's urine specific gravity time series over the course of one year, in accordance with an embodiment of the present disclosure;

FIG. 4 depicts aspects of the statistical performance of an example embodiment that is actually reduced to practice for predicting future urolithiasis for a patient, including a receiver operating characteristic (ROC) curve;

FIGS. 5A-5C illustratively provide an example embodiment of a computer program routine for generating a forecast of urolithiasis for a patient over a future time, in accordance with an embodiment of this disclosure and further described in connection to FIG. 2; and

FIG. 6 illustratively provides an example computer program routine for evaluating performance of an example embodiment reduced to practice and generating the ROC of FIG. 4.

### DETAILED DESCRIPTION

The subject matter of the present disclosure is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

As one skilled in the art will appreciate, embodiments of this disclosure may be embodied as, among other things: a method, system, or set of instructions embodied on one or more computer readable media. Accordingly, the embodiments may take the form of a hardware embodiment, a software embodiment, or an embodiment combining software and hardware. One embodiment takes the form of a computer-program product that includes computer-usable instructions embodied on one or more computer readable media.

Computer-readable media include both volatile and nonvolatile media, removable and nonremovable media, and contemplate media readable by a database, a switch, and various other network devices. By way of example, and not limitation, computer-readable media comprise media implemented in any method or technology for storing information, including computer-storage media and communications media. Examples of stored information include computer-useable instructions, data structures, program modules, and other data representations. Computer storage media examples include, but are not limited to information-delivery media, RAM, ROM, EEPROM, flash memory or other non-transitory memory technology, CD-ROM, digital versatile discs (DVD), holographic media or other optical disc storage, magnetic cassettes, magnetic tape, magnetic disk storage, other magnetic storage devices, and other computer hardware or non-transitory storage devices. These technologies can store data momentarily, temporarily, or permanently.

Aspects of the technology described herein provide improved decision support for the healthcare of patients prone to recurrent urolithiasis. In an embodiment, a decision support tool is provided that determines a numerical probability of future recurrent urolithiasis in a patient based on temporal patterns in urinalysis parameters of the patient. A time series Hölder exponent and recurrence quantification analysis (RQA) recurrence rate may be utilized to generate a forecast of recurrent symptomatic urolithiasis for a future time, such as a multi-year time horizon. The forecast may include a statistical probability or prediction score, which may be used by the decision support tool to determine and/or invoke a particular action in response to the score. For example, based on the generated forecast, one or more actions may be carried out automatically or may be recommended, such as, without limitation, intervening in the patient's care, modifying a care program for treating the patient, automatically scheduling interventions or consultations with specialist caregivers, or generating notifications such as electronic messages, which may include recommendations, information, alerts, or alarms, based on the forecast which may be emitted or otherwise provided to the caregiver and/or to the patient.

For instance, in one embodiment an indicator of a patient's multi-year probability of deterioration may be provided and utilized to determine recommendations regarding intervention or other changes in treatments plans. Intervening actions may also include recommendations or preventive actions that may be clinically indicated and such that the patient's adherence to effective prevention or treatment regimens yields indicia of reduced risk. Some embodiments of the decision support tool may quantitatively predict whether or not temporal patterns in urinalysis parameters merit intervention to prevent formation or recurrence of kidney stones, or determining adherence to or efficacy of treatment or preventive interventions.

Embodiments of the technologies described herein improve upon conventional decision support tools for treating patients with urolithiasis and are particular suitable for human patients in whom conventional urolithiasis risk-determining technology yield excessive false-negative results. For example and as further described herein, in contrast to the prior or conventional technology, it is not necessary, in some embodiments described herein, to measure variables that have either a causal role or a protective role in the process leading to stone formation or prevention. Surrogate variables that have a strong statistical association with the process are sufficient.

Some embodiments of the technologies described herein the can utilize information determined from routine diagnostic testing provided at various medical facilities and stored electronically. Thus, in these embodiments, the present technology provides for using information derived from multiple patients, over time, and at different healthcare facilities through EHR systems. Therefore, computer functionality is improved and advanced over conventional technologies, as the computer may now make predictions for a patient with urolithiasis, which have less false-negative results than conventional methods of trying to diagnostically test each individual patient, which may or may not be initially performed on a patient based on the treating clinician's experience with such rare conditions.

According to one embodiment, as will be further described herein, systems and computerized methods are provided for tracking the clinical and physiologic status of a stone-former patient, generating a forecast of urolithiasis for the patient over a future time interval and providing computer-performed decision support, including invoking one or more actions or recommendations. In some embodiments, these systems or methods are incorporated into a decision support tool used for screening, monitoring, and/or treating the patient.

A method embodiment comprises first acquiring serial, quantitative measurements of at least one physicochemical property of the urine of a target patient from one or a plurality of physiological inputs, which may include laboratory results and/or a patient's electronic health record (“EHR”). A set or urinary variables, as described herein, are determine to be monitored and forecasts. The particular variables may be determined based on available information in the patient's EHR and/or based on available resources for acquiring the data (e.g., laboratory capabilities). Data for the variables is received a plurality of times periodically or after a passage of time thereby indicating the value of the urinary variables for the patient over time. For instance, in an embodiment, the urinary variable values are acquired after a minimum of 4 hours between measurements. The specific interval of time may be based on the particular variable being observed. Thus, some variables may require shorter time intervals between measurement.

The measurement set of urinary variable data may be used to generate a time series, based on the date-time associated with each observation or measurement. In some embodiments, a minimum number of measurements is received such that the time series is a minimum length. Similarly, the observations may occur for a minimum period of time (e.g., hours or days) until enough measurements have been obtained. For example, in an embodiment, at least 90 measurements are acquired (i.e., a time series generated from the data has at least 90 component measurements).

In some embodiments, the time series may be analyzed to determine that the measurement set does not have constant values. If it is determined that the measurement set includes constant or near-constant (or substantially similar) values, then additional measurements may be acquired (with each additional measurement acquired after a time interval t, such as 4 hours.) On the other hand, if the values of the time series measurement set are not constant, then the method may proceed to periodically compute a forecast, for the likelihood of forming symptomatic stones in the urinary tract, based on the time series of urinary variable measurements.

In particular, in an embodiment, using the time series of measurement values, Hölder exponents and an RQA rate are determined and combined to create a composite forecast. The forecast may be evaluated to determine whether it is within or outside of control limits (or an acceptable or desired range). Based on whether the forecast satisfies the control-limit threshold(s), if the forecast is within acceptable control limits, then the method may end or may continue, in which case, additional measurements or observations of the urinary variables are received from the patient. The additional measurements or observations are added to the measurement set time series and may be used to generate an updated forecast. However, if the forecast falls outside of the control-limit threshold(s), then one or more actions may be invoked, as described herein. In some embodiments, an application and graphical user interface are provided for displaying information related to the one or more actions and/or displaying aspects of the generated forecast for the target patient.

When considering monitoring and treatment scenarios of patients suffering from urolithiasis, there are some urolithiasis recurrence events that are acute, with sudden onset and no apparent antecedent abnormality or multivariate cluster of abnormalities that predict an imminent event. Fortunately, from a screening and diagnostic perspective, a majority of patients who deteriorate or who experience a recurrence have physiological indicators that can be captured or measured, such as a prodrome of urinary abnormalities for many days in advance of the onset of symptomatic urolithiasis. (As further described herein, these physiological indicators may be measured or otherwise received and used to generate a time series.) This affords a ‘window of opportunity’ sufficient for undertaking effective preventive and corrective actions and intensified monitoring so as to intervene more quickly and effectively than would otherwise tend to occur. In many instances, the prodrome involves a change in statistical relationships (e.g., utilizing an autocorrelation of one variable with itself; cross-correlations between pairs of variables; or similar procedures) that bear on the natural physiologic coupling between the organ systems and processes that give rise to the measured variables; for example, the relation of hypocitraturia to stone formation, embodied in composite functions of these variables, such as citrate*calcium or pH*uric in the context of care.

The statistical distributions of the values taken on by the terms in such derived, composite variables are both skewed and asymmetric, both under normal conditions and under various pathophysiologic conditions that give rise to actionable events that relate to medical outcomes. The practical reality, however, is that statistical tests of the goodness-of-fit of distributions to data may require a considerable number of observations in order to produce a reliable conclusion or p-value.

Major problems in delivery of safe and effective prevention and care services involve deficiencies in the quality and continuity of patient care, including the monitoring of each patient's condition over time. Despite recent advances in electronic health records (EHR) systems, the present state of the art in medical care still does not in general utilize the accruing medical record information for active, prognostic use-cases, to predict the future status or events or outcomes that are likely to materialize for the patient. Instead, in many scenarios the EHR acts mainly as a passive repository for documenting and storing the information that is generated by each provider and each department, which characterizes the current or previous status or outcomes that have already materialized.

During ongoing patient management in situations requiring monitoring for urolithiasis, each patient may over a period of time see many doctors and many nurses. Such fragmentation of responsibility for the care process challenges the ability of each provider to quickly and accurately grasp the meaning of the constellation of accumulating clinical and laboratory facts about the patient, to understand trends that may be developing in the patient's health status, and to evaluate the urgency of attention that is necessary to effectively address existing or newly developing issues or to successfully prevent potential adverse events and complications. In the case of patients with urolithiasis risk, this situation is further complicated by the fact that most urinalysis values of such patients are within normal limits. In other words, there is no obvious or remarkable abnormality that would draw attention or trigger further evaluation or action.

The consequence of relatively infrequent assessment of urine status, often in stone clinics at intervals of 6 months or more, when combined with the all-too-common fragmentation of the care process with responsibilities divided among dozens of provider personnel most of whom do not have deep or longstanding familiarity with the patient, is that unexpected stone recurrence occurs to many patients, such that acute care is required. In many such instances, the impending deterioration could have been predicted-provided that more frequent urine monitoring were acquired in advance; provided that that data were integrated into a suitably accurate personalized decision support tool and predictive model; and provided that the tool (or output of the predictive model) were effectively communicated to the providers who have the responsibility to intervene and prevent or manage the predicted risk of deterioration.

Subtle patterns in urine measurements may presage the development of symptomatic kidney stones. Frequent measurement is a strategy that has often been neglected but is now enabled by the introduction of small, comparatively inexpensive point-of-care testing devices having ergonomics and low complexity compatible with routine testing by the consumer. Many such devices today possess Bluetooth or WiFi or USB communications connectivity so that data can be easily uploaded for analysis and interpretation, and results and recommendations and be quickly presented to both the consumer and to clinicians who are responsible for their care.

The probability distributions of urine analyte signals, such as specific gravity or pH, is, in general, non-stationary. When an objective function's minimum is non-stationary, its moving average location drifts and the optimization goal is one of tracking the optimal vector on short sequences of observations or short time-scales or both. In the case of longitudinal monitoring where the status of the patient often changes relatively quickly, the optimum may drift rapidly. Further, the systems that give rise to the measured data tend to embody a chaotic, stochastic process for which least-mean-square or recursive least-square deterministic optimizer that requires estimating a derivative with respect to time does not produce forecasts of adequate accuracy. However, unlike conventional approaches, embodiments of the technology described herein can accommodate aperiodic, gappy sampling and rapid-drift non-differentiable processes. Further, these embodiments overcome certain drawbacks associated with the conventional technology by providing a means for longitudinally calculating and tracking the patient's risk of urolithiasis recurrence. For instance, some embodiments provide a predicted probability of recurrence for a known stone-former patient, as further described below.

Despite the emphasis, of the conventional technology, on stone formation under conditions of dehydration and concentrated urine of high specific gravity, stones may form even under conditions of low specific gravity. Serial biochemical measurement of urinary citrate concentration or oxalate concentration may be useful in assessing whether or not adequate citrate is present in urine, sufficient to inhibit growth of calcific stones. However, use of citrate measurements is limited by the lack of availability of dipstick or other inexpensive analytical methods that could be reliably performed by consumers. Also, hypocitraturia and hyperoxaluria is relevant only to formation of calcific stones and is unrelated to formation of uric acid or cystine or other stone types. One conventional approach has uses far-UV and FTIR spectroscopy, but these are not compatible with the requirement for low cost and modest complexity and are not relevant to ascertaining all stone types. Low pH favors formation of stones comprised of calcium oxalate, calcium phosphate, and uric acid. High pH favors struvite stone formation. Yet the precision and accuracy of colorimetric dipstick and inexpensive pH meter methods are likely inadequate for discriminating patients at risk for urolithiasis.

There is little doubt that chronic dehydration accompanied by low urine volume, urine of low pH or excessively high pH, and urine that constantly has high concentrations of solutes is lithogenic. Likewise, there is little doubt that maintaining good hydration with ample production of a dilute urine is protective against stone formation. However, these aspects do not account for the relatively common finding of symptomatic kidney stones in individuals who are well-hydrated and whose urinalyses and blood chemistries are never abnormal. The known relationships of stone formation to urine concentration and abnormal pH do not account for the fact that increasing urine output to 2.5 L/day or more only reduces recurrence of symptomatic stones by approximately 50%. Nor do they account for the fact that urine pH of healthy persons is frequently found not to differ from urine pH of stone-formers. Additionally, many persons whose long-term diurnal pattern of modest daily fluids intake causes them to be chronically dehydrated and to produce highly concentrated urine do not develop stones. In such persons, there likely are protective processes that are able to physiologically compensate for the habitual dehydration pattern.

One study instructed stone-formers to monitor their urine specific gravity with daily dipstick testing. Subjects succeeded in adhering to the monitoring regime and altering their fluid intake behavior so as to achieve higher urine production and lower specific gravity below 1.010 g/mL. Some inconvenience was remarked upon, and there was recurrence of stones in some of the subjects despite the monitoring and behavior change. A similar dipstick-based study included anecdotal remarks conjecturing the possible contributory role of nighttime production of more concentrated urine as a lithogenic “trigger”. Another study directly collected urine from the bladder and the kidney of 11 stone formers and 11 control subjects under general anesthesia, noting that, beyond theoretical considerations on lithogenesis, the absence of differences in urinalysis limits the utility of conventional analyses of freshly-voided random urine specimens in assessing lithogenic risk or in follow-up of treatment or preventive interventions for patients who are especially prone to stone recurrence. Yet another study examined spinal cord injury (SCI) patients, individuals in whom urolithiasis is more common than in non-injured persons. The occurrence of stones in SCI was not significantly related to gender, race, severity of spinal cord injury, urinary tract infection, nor urine pH.

Accordingly, recurrence of stones is not a matter of simple super-saturation of urinary solutes. The understanding of the formation of urinary stones centers around three main mechanisms: the urinary concentration of stone-forming ions, the role of promoters, and the role of inhibitors of crystal formation and crystal aggregation. With respect to the promoting activity, recent emphasis has shifted from the role of the organic matrix to that of one salt inducing by epitaxy the precipitation of another salt. Among the inhibitors, it may be necessary to distinguish between those affecting crystal formation and those affecting crystal aggregation. The main inhibitors for calcium phosphate and calcium oxalate precipitation and crystal formation are citrate, pyrophosphate, and magnesium. Inhibitors for calcium phosphate and calcium oxalate crystal aggregation are glycosaminoglycans, pyrophosphate, and citrate. Among the synthetic inhibitors, the diphosphonates are the most powerful for both processes.

Most stones contain more than one type of crystals, and some combinations, such as calcium phosphate/calcium oxalate, are more common than others. Analyses performed of different locations in a stone show substantially different compositions in a large percentage of stones, which reflects their spatial heterogeneity. Epitaxy between the crystals may play a role in growth of such stones. Close association between crystals of calcium phosphate and calcium oxalate are found in human stones and stones in other mammalian species. Crystals are associated with an organic matrix on the surface and contained copious amounts of organic material within the crystalline entities. Interface between the crystals also appears to be occupied by organic matrix. Epitaxy between various crystals, even though theoretically possible, may be unlikely in vivo. The appearance of specific crystalline combinations in stones is probably a result of the urinary environment being conducive for crystallization of those components. Heterogeneous nucleation of calcium oxalate is most probably induced by biological elements, including membranous cellular degradation products.

The risk of calcium oxalate (CaOx) crystallization at different pH levels was determined in urine from recurrent CaOx-stone formers and normal subjects. The highest crystallization risk was observed between pH 4.5 and 5.5. In the pH range 6.5-7.5, there was a marked increase in crystallization of calcium phosphate (CaP). The results suggest the beneficial effect of moderate alkalinization in terms of a reduced CaOx crystallization. Reduced CaOx crystallization occurs at the expense of an increased formation of CaP crystals. Whether this increases the risk of CaP-stone formation is not known, but the CaP crystals were usually small, at least below pH 7.5.

Typical renal stone minerals such as the calcium phosphates and oxalate hydrates may nucleate, grow, or dissolve in the fluctuating concentration conditions of the urinary tract. A study has been made of the rates of formation of these minerals under conditions in which the concentrations of ionic species were maintained constant by the potentiometrically controlled addition of lattice ions. The kinetic studies were made under conditions of low supersaturation similar to those in vivo and, in the case of calcium phosphates, at least three solid phases have been shown to participate in the overall reaction depending upon factors such as pH, nature and concentration of supporting medium, supersaturation, ionic strength, temperature, and the presence of inhibitors of crystallization. For the calcium oxalates, the mineralization process may involve the initial precipitation of less stable hydrates before conversion to the most stable whewellite. Urinary stone inhibitors may have a considerable influence in stabilizing thermodynamically less stable hydrates. One study examined epitactic relationships between hydroxyapatite (HAP) and the hydrates of calcium oxalate from a crystallographic point of view. It also examined the growth of HAP on the calcium oxalate surfaces at a constant supersaturation, maintained by the controlled addition of solutions containing the lattice ions of the precipitating phase. Calcium oxalate trihydrate was the only salt that induced HAP overgrowth. The latter phase, however, was found to be a suitable substrate for the growth of calcium oxalate monohydrate.

Two theories explain urate's apparent stone-promoting effect. The first proposes that urinary urate crystals promote CaOx precipitation by the phenomenon of epitaxy; the second hypothesis is that colloidal particles of urate reduce the inhibitory activity of urinary glycosaminoglycans (GAGs) which normally prevent the crystallization of CaOx. Recent research indicates that at normal physiological pH values dissolved urate directly promotes CaOx precipitation by the classic ‘salting-out’ effect by enhancing nucleation, growth and aggregation of CaOx crystals. Therefore a suggested beneficial effect of allopurinol in reducing CaOx stone recurrences may be attributed to its lowering the urinary output of urate and thereby reducing the probability that CaOx will be salted out of urine, rather than to epitaxy or inactivation of urinary GAGs. At low ionic strength, stabilization of water hydration shells of calcium ions by non-paired electrolytes leads to a reduction in the calcite dissolution rate compared to pure water. At high ionic strength, salts with a common anion yield similar dissolution rates. Multi-layered biominerals, also known as Randall's plaques, consist of alternating strata of organic-and inorganic-dense materials serve as attachment sites for calcium oxalate overgrowths to form into kidney stones.

Normal urine is periodically supersaturated with respect to lithogenic solutes and, not surprisingly, prior theory of stone formation has assumed equilibrium solubility-product conditions that characterize this supersaturation. However, non-equilibrium conditions may prevail in the tubules and collecting system and may contribute to nucleation and lamellar accretion onto nidi and stones. In dilute urine with concentrations intermittently below the solubility-product of calcium oxalate or the solubility-product of calcium phosphate, the stones do not dissolve. Instead, the surface layer may stabilize, adsorbing glycosaminoglycans or other solutes or changing hydrate composition or crystal packing. Then, when urine again becomes more concentrated or pH changes or other composition changes ensue (covered by a layer of urine proteins such as THP and OPN, or a layer of urate on calcific stones), heteroepitaxial deposition resumes.

Although fluctuating supersaturation and decreased ambient crystallization inhibitors can account for crystal nucleation and crystalluria, additional mechanisms are needed to explain the sporadic abnormalities and ongoing accretion that result in symptomatic stones. An approach that uses time series analysis of temporal patterns, as utilized by embodiments described herein, can detect or predict the occurrence of conditions under which such mechanisms are active. It has been shown that hypercalciuria and other solute saturation-related abnormalities are present only in a small percentage of cases (˜25%) on any 24-hour urine specimen. Those same abnormalities are present in about 15% of persons who do not have a history of urolithiasis. Thus, there is a need for better prognostics/diagnostics, including decision support technology, having improved accuracy sufficient to guide prescribing pharmacologic preventive measures, dietary, or lifestyle changes. The fact that stone formation is a slow process where the rate of accretion of material onto existing stone nidus exceed the rate of dissolution over many weeks to years. Serial daily testing can better characterize the intermittent and time-average or cumulative risk.

A cross-sectional study was performed of 24-h urine excretion and the risk of kidney stone formation in 3,350 men and women, of whom 2,237 had a history of nephrolithiasis. After adjusting for other urinary factors, urinary uric acid had a significant inverse association with stone formation in men, a marginal inverse association with risk in younger women, and no association in older women. The risk of stone formation in men and women significantly rose with increasing urine calcium and oxalate, and significantly decreased with increasing citrate and urine volume, with the change in risk beginning below the traditional normal thresholds. Other urinary factors were also associated with risk, but this varied by age and gender. The study did not support the prevailing belief that higher urine uric acid excretion increases the risk for calcium oxalate stone formation.

The conventional technology utilizing a 24-hour urine test has several limitations including the difficulty of achieving adherence to collection protocol, complexity of interpretation, need for repeat collections, inability to predict stone recurrence with individual parameters and supersaturation values, unclear rationale of laboratory cutoff values, and difficulty with determining collection adequacy. For instance, only one prospective trial has compared selective dietary recommendations based on 24-hour urine collection results versus general dietary instructions.

Another limitation of the conventional technology is that it is primarily relevant at the population level. Increasing body mass index is related to several risk factors for urinary stone disease, including increasing urine sodium and decreasing pH in men and increasing urine uric acid, sodium, decreasing urine citrate in women, elevated BMI, anxiety states, sedentariness or low daily step count, as well as diabetes or gout or other metabolic disorders. While these parameters are instructive in regards to population-level policies regarding testing, they are not predictive of first-time or recurrent urolithiasis in an individual patient, nor are they actionable in terms of preventive or therapeutic interventions on an N-of-1 individual basis.

In other instances of the conventional technology, abnormalities in urinalysis variables give rise to ‘false-positive’ errors, incorrectly identifying a given patient as one in whom symptomatic urolithiasis is likely when in fact no such event occurs. In such a situation, valuable resources associated with repeated ultrasound evaluation and intensified monitoring or other interventions are misapplied. The resources are allocated to the given patient, in whom those resources are not in fact necessary and provide no benefit, and, insofar as resources are finite and in short supply, those resources are during that same time interval withheld from other patients, for whom the resources might have provided greater value and benefit. Thus, a significant limitation of the conventional technology is that it suffers from limited statistical sensitivity and specificity, with substantial false-negative and false-positive rates. Most of the conventional diagnostic/prognostic implementations only address certain solute concentrations and chemical solubility-product indices thereof. Such implementations therefore fail to identify stone formation mechanisms that do not involve the measured solutes.

Yet another limitation of the conventional technology is that it typically relies upon measurements that are often measured with insufficient precision for achieving predictive power and accuracy. A further limitation of the conventional technology is that its processes are performed only infrequently and, as such, characterize the urine's lithogenicity at only the moment in time when the urine specimen was obtained, whereas the true risk is manifest over a period of months to years, periods when no testing is performed.

A yet further limitation of the conventional technology is that its analytical methods require extensive pre-analytical manipulation, such as acidification with 2N hydrochloric acid or other agents. Some assay methods require long elapsed time periods sufficient for chemical reaction or precipitation, or entail considerable within-and between-observer variability associated with tube-shaking, container-tapping, pellet-washing, or other manipulations prior to analysis. Some methods used in the conventional technology involve high-performance liquid chromatography (HPLC), Fourier-transform infrared (FTIR) spectroscopy, or other means that require expensive analytical equipment, reagents, and expert analysts to perform and interpret the testing.

A still further limitation of the conventional technology is that its predictive capability is not associated with a particular time frame such as would facilitate making the results actionable in a manner that would promote the patient's psychological motivation to adhere to prescribed therapy. If no action that the patient takes produces a noticeable change in the residual risk or likelihood of the event's future occurrence, then motivation and adherence are diminished.

But some embodiments of the technology described herein may optionally utilize automated storage and processing of recorded values on a handheld measuring device or smartphone, plus transmission of data from such devices via wired (such as LAN or telephone) or wireless means (such as Bluetooth, Wifi LAN, or cellular telephony) to remote storage and processing facilities. In this way, patients can then readily and more immediately manage their risk of recurrent urolithiasis by adapting their fluids intake, modifying their urine by means of citrate therapy, using a phosphate binder or oxalate binder or xanthine oxidase inhibitor therapy, modifying their intake of oxalate-rich or nucleic-acid-rich foods, or supplementing their diet with probiotics, such as Oxalobacter formigenes. Moreover, patients are more likely to adhere to a measurement regimen if they are not required to perform complex or long-duration urine collections, undertake careful dilutions or reagent additions to perform the testing, or transcribe testing results into other records. Such patients can with a simple handheld or dipstick measurement device and method likewise avoid pre-analytic errors resulting from urine collection (including failure to obtain specimens at the desired times, or failure to keep the specimen until it can be tested), transport, and storage.

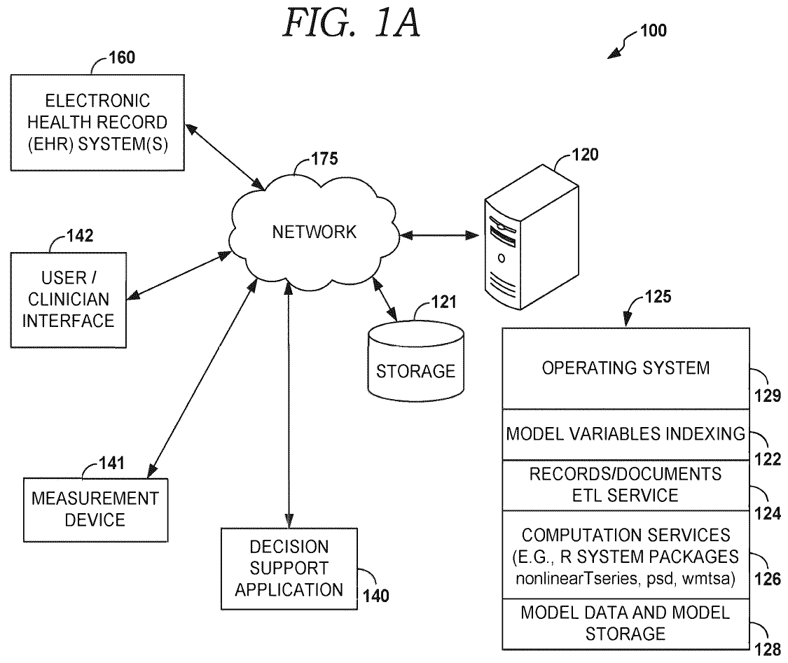

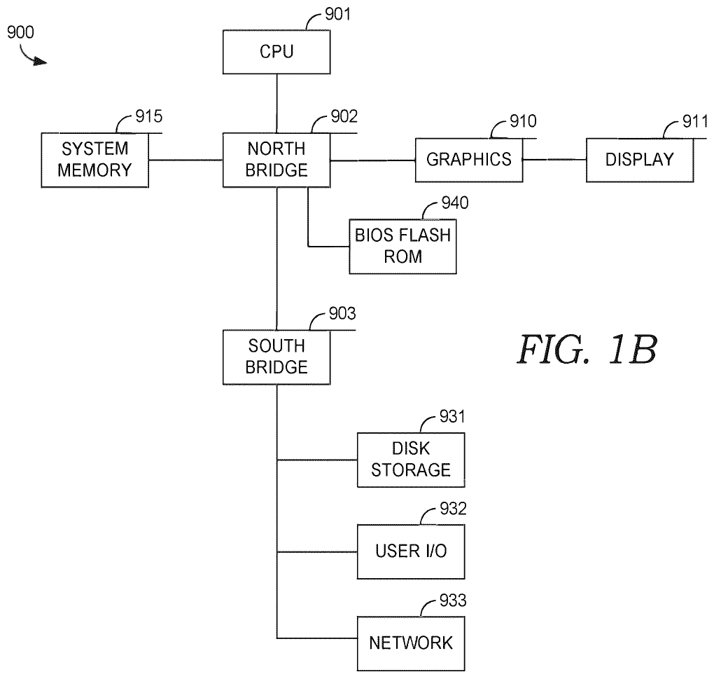

Referring now to the drawings in general, and initially to FIG. 1A in particular, an aspect of an operating environment 100 is provided suitable for practicing an embodiment of the technologies described herein. We show certain items in block-diagram form more for being able to reference something consistent with the nature of a patent specification than to imply that a certain component is or is not part of a certain device. Similarly, although some items are depicted in the singular form, plural items are contemplated as well (e.g., what is shown as one data store might really be multiple data-stores distributed across multiple locations). But showing every variation of each item might obscure aspects of the technology. Thus for readability, we show and reference items in the singular (while fully contemplating, where applicable, the plural).

As shown in FIG. 1, a block diagram is provided showing aspects of an example computing system architecture suitable for implementing an embodiment of this disclosure and designated generally as example operating environment 100. Example operating environment 100 provides an aspect of a computerized system for compiling and/or running aspects of this disclosure including collecting and analyzing unstructured text data from electronic health record(s), which may include claims data, to assess the texts as to topical or concept-oriented expressions they contain that are statistically similar to those associated with various clinical conditions or diagnoses; to identify which condition-or diagnosis-oriented clusters the present texts most closely resemble, if any; and to notify the responsible clinicians of those determinations, suggesting consideration of those conditions or diagnoses as part of the constellation of differential diagnoses pertinent to the management of the current patient.

Operating environment 100 is one example of a suitable environment and system architecture for implementing an embodiment of the disclosure. Other arrangements and elements can be used in addition to or instead of those shown, and some elements may be omitted altogether for the sake of clarity. Further, as with operating environment 100, many of the elements described herein are functional entities that may be implemented as discrete or distributed components or in conjunction with other components, and in any suitable combination and location. As described above, some embodiments may be implemented as a system, comprising one or more computers and associated network and equipment, upon which a method or computer software application is executed. Accordingly, aspects of the present disclosure may take the form of an embodiment combining software and hardware aspects that may all generally be referred to herein as a “module” or “system.” Further, the methods of the present disclosure may take the form of a computer application embodied in computer readable media having machine-readable application software embodied thereon. In this regard, a machine-readable storage media may be any tangible medium that can contain, or store a software application for use by the computing apparatus.

Computer application software for carrying out operations for system components or steps of the methods of the present disclosure may be authored in any combination of one or more programming languages, including an object-oriented programming language such as Java, Python, R, or C++ or the like. Alternatively, the application software may be authored in any or a combination of traditional non-object-oriented languages such as C or Fortran. The application may execute entirely on the user's computer as an independent software package, or partly on the user's computer in concert with other connected co-located computers or servers, or partly on the user's computer and partly on one or more remote computers, or entirely on a remote computer or collection of computers. In the latter cases, the remote computers may be connected to the user's computer through any type of network, including a local area network (LAN) or a wide area network (WAN), or the connection may be made to an external computer (for example, via the internet using an Internet Service Provider or ISP) or an arbitrary, geographically-distributed, federated system of computers, such as a cloud-based system.

Moreover, the components of operating environment 100, functions performed by these components, or services carried out by these components may be implemented at appropriate abstraction layer(s) such as the operating system layer, application layer, hardware layer, etc., of the computing system(s). Alternatively, or in addition, the functionality of these components and/or the embodiments described herein can be performed, at least in part, by one or more hardware logic components. For example, and without limitation, illustrative types of hardware logic components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. Additionally, although functionality is described herein with regards to specific components shown in example system 200, it is contemplated that in some embodiments functionality of these components can be shared or distributed across other components.

Environment 100 includes one or more electronic health record (EHR) systems, such as EHR system(s) 160 communicatively coupled to network 175, which is communicatively coupled to computer system 120. In some embodiments, components of environment 100 that are shown as distinct components may be embodied as part of or within other components of environment 100. For example, EHR system(s) 160 may comprise one or a plurality of EHR systems such as hospital EHR systems, health information exchange EHR systems, clinical genetics/genomics systems, ambulatory clinic EHR systems, psychiatry/neurology EHR systems, insurance, collections or claims records systems; and may be implemented in or as a part of computer system 120. Similarly, EHR system(s) 160 may perform functions for two or more of types of EHR systems (not shown). EHR system(s) 160 also may include records of urinalysis variables obtained via one or more measurement apparati, tests, or screenings, such as measurement device 141. may perform functions for two or more of types of EHR systems (not shown). In an embodiment, EHR system(s) 160 includes historical claims data for health services, apportionment data, and related health services financial data.

In some embodiments of the technologies described herein, aspects of decision support for patients prone to urolithiasis may utilize data about a population of patients derived from patient EHR or other records information. In particular, presently certain data warehouses are created for purposes of public health and observational research purposes and are derived from electronic health records repositories in such a way that they are de-identified so as to comply with applicable confidentiality laws and regulations. The Cerner Health Facts™ data warehouse is such a system that has been curated for more than 15 years. It comprises a large ‘transaction database’ where each entry corresponds to a patient's ‘basket’ (a collection of items recorded or transacted at points in time during episodes of care services provisioning in the contributing health care institutions). Each database entry is ordered by the date-time of the transaction. Transaction sequencing is implemented by grouping medical events occurring in the same ‘epoch’ for the same patient together into ‘baskets’ and ordering the ‘baskets’ of each patient by the date-time stamps where the events occurred. Epoch durations may differ according to the age of the patient, or the acute or chronic nature of the health conditions that pertain to the patient, or the rate of change of the severity of the health conditions, or other factors, Epoch durations may be as short as a few minutes (as in critical care ICU or operating room contexts) or may be as long as 10 years or more (as in chronic ambulatory care-sensitive conditions, ACSCs).

Continuing with FIG. 1A, network 175 may comprise the Internet, and/or one or more public networks, private networks, other communications networks such as a cellular network, or similar network(s) for facilitating communication among devices connected through the network. In some embodiments, network 175 may be determined based on factors such as the source and destination of the information communicated over network 175, the path between the source and destination, or the nature of the information. For example, intra-organization or internal communication may use a private network or virtual private network (VPN). Moreover, in some embodiments items shown communicatively coupled to network 175 may be directly communicatively coupled to other items shown communicatively coupled to network 175.

In some embodiments, operating environment 100 may include a firewall (not shown) between a first component and network 175. In such embodiments, the firewall may reside on a second component located between the first component and network 175, such as on a server (not shown), or reside on another component within network 175, or may reside on or as part of the first component.

Embodiments of electronic health record (EHR) system(s) 160 include one or more data stores of health-related records, which may be stored on storage 121, and may further include one or more computers or servers that facilitate the storing and retrieval of the health records. In some embodiments, EHR system(s) 160 and/or other records systems may be implemented as a cloud-based platform or may be distributed across multiple physical locations. EHR system(s) 160 may further include record systems, which store real-time or near real-time patient (or user) information, such as wearable sensor or monitor, bedside, laboratory, or in-home patient monitors or sensors, for example, such as measurement device 141.

Example operating environment 100 further includes a user/clinician interface 142 and decision support application 140, each communicatively coupled through network 175 to an EHR system 160. Although environment 100 depicts an indirect communicative coupling between interface 142 and application 140 with EHR system 160 through network 175, it is contemplated that an embodiment of interface 142 or application 140 are communicatively coupled to EHR system 160 directly. An embodiment of decision support application 140 comprises a software application or set of applications (which may include programs, routines, functions, or computer-performed services) residing on a client computing device (or distributed in the cloud and on a client computing device) such as a personal computer, laptop, smartphone, tablet, or mobile computing device. In an embodiment, the application is a Web-based application or applet, and may be used to provide or manage user services provided by an embodiment of the technologies described herein, which may be used by a caregiver or screener to provide, for example, information about the likelihood of a specific patient or population of patients to experience urolithiasis recurrence at a future time. In some embodiments, application 140 includes or is incorporated into a computerized decision support tool, as described herein. Further, some embodiments of application 140 utilize user/clinician interface 142.

In some embodiments, application 140 and/or interface 142 facilitates accessing and receiving information from a user or health care provider about a specific patient or set of patients, according to the embodiments presented herein. Embodiments of application 140 also may facilitate accessing and receiving information from a user or health care provider about a specific patient, caregiver, or population including historical data; health care resource data; variables measurements, timeseries, and predictions (including plotting or displaying the determined outcome and/or issuing an alert) described herein; or other health-related information, and facilitates the display of results, recommendations, or orders, for example. In an embodiment, application 140 also facilitates receiving orders, staffing scheduling, or queries from a user, based on the results of monitoring and/or forecasted outputs, which may in some embodiments utilize user interface 142. Decision-Support application 140 may also be used for providing diagnostic services or evaluation of the performance of various embodiments.

In some embodiments, user/clinician interface 142 may be used with application 140, such as described above. One embodiment of user/clinician interface 142 comprises a user interface that may be used to facilitate access by a user (including a clinician/caregiver such as a medical or psychiatric caregiver or the like) to a score or prediction determined according to the technologies described herein, including information indicating a likelihood that a patient will experience urolithiasis over a future time interval or other aspects of forecasts for urolithiasis described herein. One embodiment of interface 142 takes the form of a graphical user interface and application, which may be embodied as a software application (e.g., decision support application 140) operating on one or more mobile computing devices, tablets, smartphones, front-end terminals in communication with back-end computing systems, laptops, or other computing devices. In an embodiment, the application includes the PowerChart® software manufactured by Cerner Corporation. In an embodiment, interface 142 includes a Web-based application (which may take the form of an applet or app) or set of applications usable to manage user services provided by an embodiment of the technologies described herein.

In some embodiments, interface 142 may facilitate providing the output of the determined forecast(s), probabilities (or score), recommendations, scheduling orders, providing instructions, or outputs of other actions described herein, as well as logging and/or receiving other feedback from the user/caregiver, in some embodiments. In an embodiment, interface 142 also facilitates receiving orders for the patient from the clinician/user, based on the results of monitoring and predictions. Interface 142 also may be used for providing diagnostic services or evaluation of the performance of various embodiments.

Example operating environment 100 includes measurement device 141 communicatively coupled through network 175 to an EHR system 160. The term measurement is used broadly herein, and it is contemplated that in some embodiments, measurement device 141 may not perform measurement but may receive information about physiological parameters (such as urinalysis variables) which may be measured, observed, or otherwise recorded. Embodiments of measurement device 141 may comprise one or more sensors, such as sensor(s), an interface component, and/or processing/communications component (not shown). For example, in one embodiment actually reduced to practice and described below, measurement device 141 comprises an Atago Model 3749 hand-held pen digital refractometer with 4-digit precision for urine specific gravity measurements. Embodiments of measurement device 141 may store user-derived data locally or communicate data over network 175 to be stored remotely. Some embodiments of measurement device 141 include a monitor interface, which may be embodied as I/O such as buttons and sounds emitted from the measurement device 141, its firmware or software application or app operating on a user's mobile device or computer system 120, and in an embodiment may facilitate uploading of measured (or recorded, or otherwise received) information from measurement device 141 to computer system 120.

Additionally, some embodiments of measurement device 141 include functionality for processing user-derived information locally or for communicating the information to computer system 120, where it is processed. In some embodiments, the processing may be carried out or facilitated by one or more software agents, as described below. In some embodiments the processing functionality, performed on measurement device 141 and/or computer system 120 includes pre-processing and/or signal conditioning, such as removing noise or erroneous information.

Example operating environment 100 further includes computer system 120, which may take the form of one or more servers, and which is communicatively coupled through network 175 to EHR system 160, and storage 121.

Computer system 120 comprises one or more processors operable to receive instructions and process them accordingly, and may be embodied as a single computing device or multiple computing devices communicatively coupled to each other. In one embodiment, processing actions performed by system 120 are distributed among multiple locations such as one or more local clients and one or more remote servers, and may be distributed across the other components of example operating environment 100. For example, aspects of application 140 or interface 142 may operate on or utilize computer system 120. Similarly, a portion of computing system 120 may be embodied on user interface 142, application 140, and/or EHR system(s) 160. In one embodiment, system 120 comprises one or more computing devices, such as a server, desktop computer, laptop, or tablet, cloud-computing device or distributed computing architecture, a portable computing device such as a laptop, tablet, ultra-mobile P.C., or a mobile phone.

Embodiments of computer system 120 include computer software stack 125, which in some embodiments operates in the cloud, as a distributed system on a virtualization layer within computer system 120, and includes operating system 129. Operating system 129 may be implemented as a platform in the cloud, and which is capable of hosting a number of services such as 122, 124, 126, and 128. Some embodiments of operating system 129 comprise a distributed adaptive agent operating system. Embodiments of services 122, 124, 126, and 128 run as a local services or may be distributed across one or more components of operating environment 100, in the cloud, on one or more personal computers or servers such as system 120, and/or a computing device running interface 142 or application 140. In some embodiments, interface 142 and/or application 140 operate in conjunction with software stack 125.

In embodiments, model variables indexing service 122 and records/documents ETL service 124 provide services that facilitate retrieving patient physiological variables, which may include frequent item sets, extracting database records, and cleaning the values of variables in records. For example, services 122 and/or 124 may perform functions for synonymic discovery, indexing or mapping variables in records, or mapping disparate health systems' ontologies, such as determining that a particular medication frequency of a first record system is the same as another record system. In some embodiments, these services may invoke computation services 126.

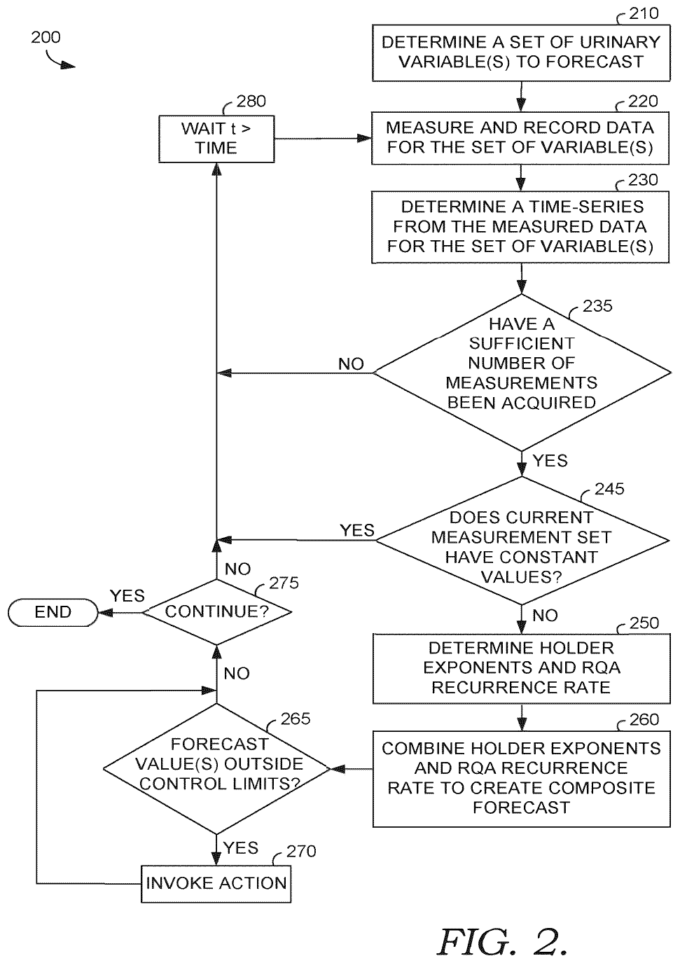

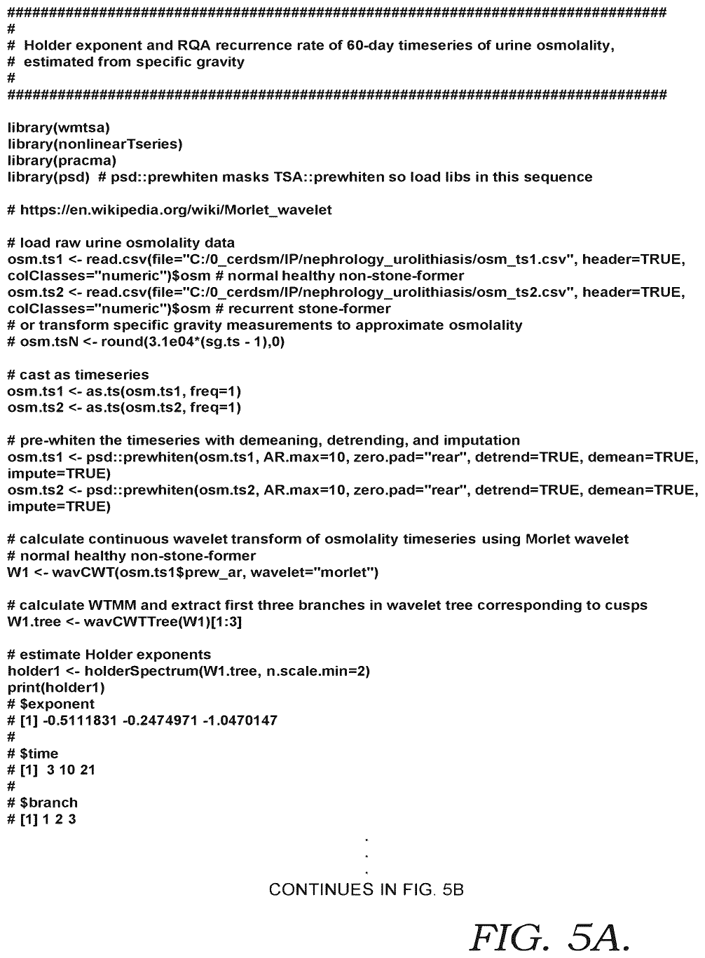

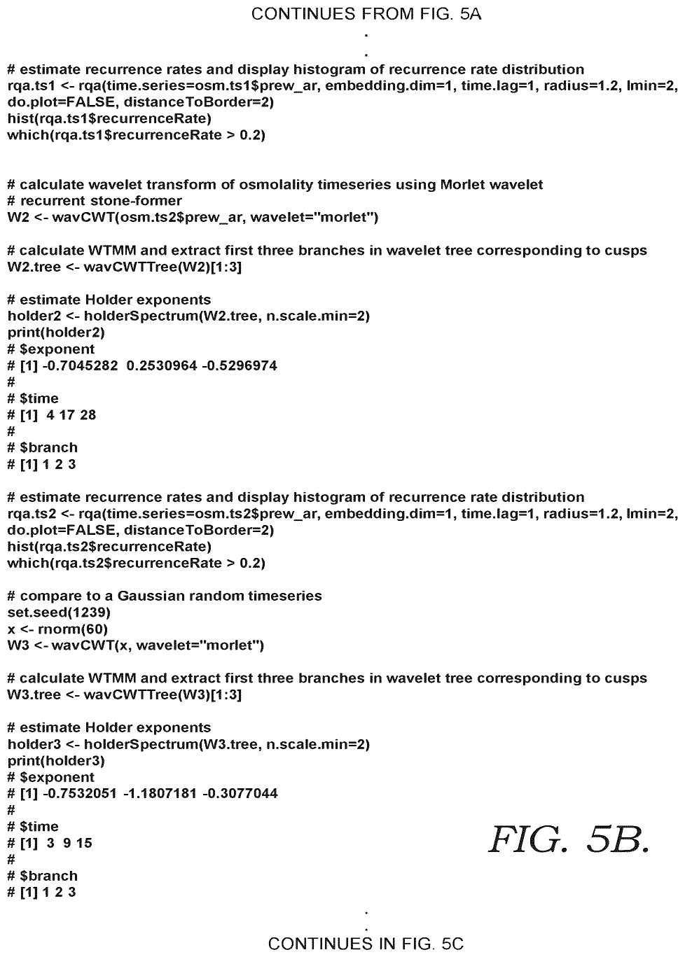

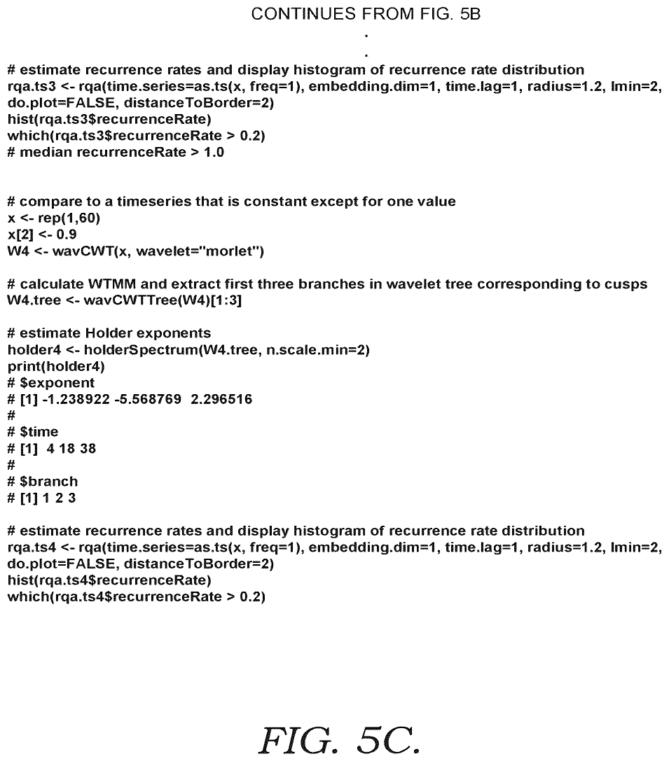

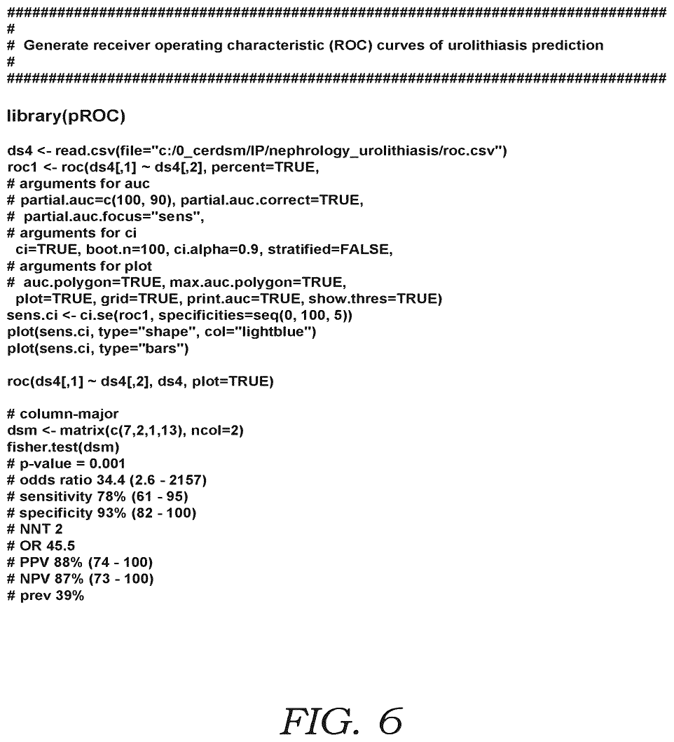

Computation services 126 may perform statistical or computing operations, and may include statistical calculation packages such as, in one embodiment, the R system (the R-project for Statistical Computing, which supports R-packages or modules tailored for specific statistical operations, and which is accessible through the Comprehensive R Archive Network (CRAN) at http://cran.r-project.org) or similar services, and R-system modules or packages such as packages nonlinearTseries, for nonlinear time series analysis including Recurrence Quantification Analysis (RQA); psd, for performing power spectral density estimates; and wmtsa, for performing wavelet methods for time series analysis. Computation services 126 also may include natural language processing services (not shown) such as Discern nCode™ developed by Cerner Corporation, or similar services. In an embodiment, computation services 126 include the services or routines, which may be embodied as one or more software agents or computer software routines such as the example embodiments of computer program routines illustratively provided in FIGS. 5A-5C and 6. Computation services 126 also may include services or routines for utilizing one or more prediction models such as described in connection to FIG. 2 and the example computer program routines illustratively provided in FIGS. 5A-5C. In some embodiments, computation services 126 use EHR system(s) 160, model data and model storage services 128, and/or other components of example operating environment 100, and may also include services to facilitate receiving and/or pre-processing physiological data. Model data and model storage services 128 may be utilized to perform services for facilitating storage, retrieval, and implementation of the forecasting models described herein and of the data used in the models.

Some embodiments of stack 125 may further comprise services for utilizing an Apache Hadoop and Hbase framework (not shown), or similar frameworks operable for providing a distributed file system, and which in some embodiments facilitate provide access to cloud-based services such as those provided by Cerner Healthe Intent®. Additionally, some embodiments of stack 125 may further comprise one or more services stream processing service(s) (not shown). For example, such stream processing service(s) may be embodied using IBM InfoSphere stream processing platform, Twitter Storm stream processing, Ptolemy or Kepler stream processing software, or similar complex event processing (CEP) platforms, frameworks, or services, which may include the user of multiple such stream processing services (in parallel, serially, or operating independently). Some embodiments of the technology disclosed herein also may be used in conjunction with Cerner Millennium®, Cerner CareAware® (including CareAware iBus®), Cerner CareCompass®, or similar products and services.

Example operating environment 100 also includes storage 121 (or data store 121), which in some embodiments includes patient data for a candidate or target patient (or information for multiple patients), including raw and processed patient data; variables associated with patient recommendations; recommendation knowledge base; recommendation rules; recommendations; recommendation update statistics; an operational data store, which stores events, frequent itemsets (such as “X often happens with Y”, for example), and item sets index information; association rulebases; agent libraries, solvers and solver libraries, and other similar information including data and computer-usable instructions; patient-derived data; and health care provider information, for example. It is contemplated that the term data includes any information that can be stored in a computer-storage device or system, such as user-derived data, computer usable instructions, software applications, or other information. In some embodiments, data store 121 comprises the data store(s) associated with EHR system 160. Further, although depicted as a single storage data store, data store 121 may comprise one or more data stores, or may be in the cloud.

Turning briefly now to FIG. 1B, there is shown one example embodiment of computing system 900 that has software instructions for storage of data and programs in computer-readable media. Computing system 900 is representative of a system architecture that is suitable for computer systems such as computing system 120. One or more CPUs such as 901, have internal memory for storage and couple to the north bridge device 902, allowing CPU 901 to store instructions and data elements in system memory 915, or memory associated with graphics card 910, which is coupled to display 911. Bios flash ROM 940 couples to north bridge device 902. South bridge device 903 connects to north Bridge device 902 allowing CPU 901 to store instructions and data elements in disk storage 931 such as a fixed disk or USB disk, or to make use of network 933 for remote storage. User I/O device 932 such as a communication device, a mouse, a touch screen, a joystick, a stylus or touch-stick, a trackball, or keyboard, couples to CPU 901 through south bridge 903 as well. The system architecture depicted in FIG. 1B is provided as one example of any number of suitable computer architectures, such as computing architectures that support local, distributed, or cloud-based software platforms, and are suitable for supporting computing system 120.

Returning to FIG. 1A, in some embodiments, computer system 120 is a computing system made up of one or more computing devices. In some embodiments, computer system 120 includes one or more software agents, and in an embodiment includes an adaptive multi-agent operating system, but it will be appreciated that computer system 120 may also take the form of an adaptive single agent system or a non-agent system. Computer system 120 may be a distributed computing system, a data processing system, a centralized computing system, a single computer such as a desktop or laptop computer or a networked computing system.

Turning now to FIG. 2A, a flow diagram is provided that illustrates a method 200 for generating a forecast of urolithiasis for a patient over a future time interval. At a high level, and a described above, embodiments of the technologies described herein may first determine Hölder exponents and/or RQA recurrence rate using a time series of urinalysis variable values, and then use the Hölder exponents and RQA recurrence rate to generate a forecast representing a likelihood of urolithiasis recurrence (such as a probability of recurrent symptomatic urolithiasis for a multi-year time horizon). With reference to FIG. 2, Hölder exponents may be used for detecting singularities in a signal by examining the evolution of the modulus maxima of the wavelet transform across 2 or more timescales. The decay of the maxima line can then be used to determine the regularity of the signal at the given time point or interval. A less complex means of extracting the maxima line is to simply examine at the decay of the wavelet modulus across the scales for each time interval or segment. Segments where large changes occur in the signal (such as cusp-type singularities) have large coefficients at all the different scales, which is consistent with little decay. Recurrence Quantification Analysis (RQA) may be used as a complementary technique for the nonlinear analysis of time series that allows to quantify the number and duration of the recurrences in the phase space that a time series comprises.

The Hölder exponent provides a means for ascertaining the regularity of a signal, such that the regularity identifies to what order a function is differentiable. For instance, if a signal f(t) is differentiable at time t, it has a Hölder exponent of +1.0. If the signal is discontinuous but bounded in the neighborhood of t, such as a step function, then the Höolder exponent is 0.0. The Dirac delta function at time t has a Hölder exponent of −1.0 since it is unbounded at t. From these examples, it can be seen that there is a relationship between the Hölder exponent of a function and its derivatives. Taking the derivative of a function decreases the regularity of the function's Holder exponent by 1 and integrating increases the regularity of the function's Hölder exponent by 1. When applied to the context of urolithiasis, the urinary osmolality time series of persons who are recurrent stone-formers tend to have at least one Hölder exponent that is significantly greater than zero for at least one segment of daily urine osmolality measurements time series at least 90 days in length. Possibly, under transient conditions of saturation or supersaturation that dispose toward crystal formation or accretion of material on existing stones such persons have a greater-than-normal tendency for that condition to persist for a time sufficient to favor stone formation. RQA analysis of such patients shows one or more recurrence rate values that are greater than 0.2. The approximate entropy and Hurst exponent values do not appear to be statistically significantly different from those of time series of stone-formers.

By contrast, in many instances we have discovered that persons who do not form stones tend to have Hölder exponents negative (less than zero) in all segments of daily urine osmolality measurements time series of sufficient length, (such as at least 90 days in length, in an embodiment). Moreover, the time series of such non-stone-forming persons are highly irregular in all time segments, reflecting a temporal responsiveness and diversity of renal function associated with the ability to produce concentrated and dilute urine and to compensate for fluctuating perfusion and filtrate composition over comparatively short time intervals. Possibly, under transient conditions of saturation with respect to certain solutes such persons' urine is likely to soon again become unsaturated, such that any crystals that formed or any accretion that occurred onto existing crystals during the interval of saturation are dissolved and excreted. Furthermore, RQA analysis of non-stone-formers shows all recurrence rates is discovered to have values less than 0.2. Utilizing these discoveries, an improved decision support technology may be provided for screening, monitoring, and/or treating patients prone to urolithiasis.

Some embodiments of method 200 may include determining a risk of urolithiasis recurrence. A forecast or future state may be determined using a time series to derive a model, and then applying the model to recent or last values of the time series, in order to extrapolate past observations of values into likely future values. By way of example and not limitations, parametric methods for estimating probabilities from time series include logistic regression, Cox proportional hazards regression, conditional proportional hazards regression, Weibull regression, Poisson regression, ARMA, ARIMA, and ARFIMA regression, log-Pearson Type 3 distribution (3-parameter gamma) regression, Generalized Extreme Value regression, Fréchet regression, and log-logistic accelerated failure time Generalized Additive Models for Location, Scale and Shape (GAMLSS). Non-parametric methods include random survival forests and survival trees, for example. In some embodiments, the forecasting uses moving-averages techniques, random-walk and trend models, exponential smoothing, state-space modeling, vector autoregressive models, cointegrated and/or causal models. Neural and/or fuzzy networks, data mining and rule-based techniques are also suitable techniques used in some embodiments of time series forecasting.

Accordingly, example method 200 comprises acquiring serial, quantitative measurements of at least one physicochemical property of the urine of a target patient from one or a plurality of physiological inputs, which may include laboratory results and/or a patient's electronic health record (“EHR”). At step 210, a set or urinary variables are determined to be monitored and forecasts. In one embodiment, the set of urinary variables comprises urine osmolality (which may be estimated or derived from specific gravity measurements of urine). Other urinary analytes, as described herein, also may be utilized. In some embodiments, the urinary variables in step 210 may be identified by a clinician or and provided via a particular computer-program routine or entered in via interface 142, for example. Further, in some embodiments, the particular urinary variables may be determined based on the target patient or characteristics of the forecast to be generated (such as the time horizon or prediction confidence). Still further, in some embodiments, the particular variables determine din step 210 may be determined based on available information in the patient's EHR and/or based on available resources for acquiring the data (e.g., laboratory capabilities).

At step 220, measure and record data for the set of variables. In embodiments of step 220, measurement data for the variables is received for a plurality of times periodically (or after a passage of time between each measurement) thereby indicating the value of the urinary variables for the patient over time. For instance, in an embodiment, the urinary variable values are acquired after a minimum of 4 hours between measurements. The specific interval of time may be based on the particular variable being observed. Thus, some variables may require shorter time intervals between measurement.

At step 230, determine a time-series from the measured data for the set of variables. The measurement set of urinary variable data may be used to generate a time series, based on the date-time associated with each observation or measurement. In some embodiments, a minimum number of measurements is received such that the time series is a minimum length. Similarly, the observations may occur for a minimum period of time (e.g., hours or days) until enough measurements have been obtained. For example, in an embodiment, at least 60 or 90 measurements are acquired (i.e., a time series generated from the data has at least 90 component measurements) each having minimum time interval t between each measurement or observation, such as hour hours. In another embodiment, measurements are acquired for a minimum number of days (such as 30, 60, or 90 days). At step 235, method 200 determine did a sufficient number of measurements have been acquired. If not, then method 200 proceeds to step 280, where a minimum about of time t transpires (e.g., a time interval of 4 hours or of sufficient length to indicate a change in the particular urinary variables being measured). Method 200 then goes back to step 220 to acquire an additional measurement. But, returning to step 235, if a sufficient number of measurements have been acquired, then method 200 proceeds to step 245.

At step 245, determine whether the current measurement set has constant values. In some embodiments, the time series may be analyzed to determine that the measurement set does not have constant values. If it is determined that the measurement set includes constant or near-constant (or substantially similar) values, then additional measurements may be acquired (with each additional measurement acquired after a time interval t, such as 4 hours), and method 200 proceeds to step 280 and then back to step 220. On the other hand, if the values of the time series measurement set are not constant or substantially constant, then the method may proceed to step 250 (and beyond) to periodically compute a forecast, for the likelihood of forming symptomatic stones in the urinary tract, based on the time series of urinary variable measurements.

At step 250, determine Hölder exponents and RQA rate using the time series measurement values. In some embodiments of step 250, the Hölder exponents are determined by estimation. Some embodiments of step 250 (and other steps of method 200) utilize computation services 126 (FIG. 1), including R System package nonlinearTseries, psd, and wmtsa. In particular, aspects of method 200 may be carried out using the example computer program provided in FIGS. 5A-5C. At step 260, the Hölder exponents and RQA recurrence rate determined in step 250 are combined to create a composite forecast. In one embodiment of step 260, a logistic regression model is created utilizing the largest Hölder exponent and the RQA recurrence rate as independent (predictor) variables for a prediction time horizon. The time horizon may be range from 6 months to 3-5 years in some embodiments. In other embodiments of step 260, other techniques may be used to generate a model, as described above. In some embodiments, the forecast created in step 260 includes a prediction probability and/or a prediction score.

At step 265, the forecast may be evaluated to determine whether it is within or outside of control limits (or an acceptable or desired range). For instance, in an embodiment of step 265, one or more thresholds are used to evaluate the forecast. In some embodiments, the threshold(s) may be predetermined, determined empirically, or based on the specific urinary variables used to generate the forecast, or based on information about the particular patient, caregiver, a treatment context (e.g., the treatment venue, role of the caregiver, insurer, or other clinical conditions or events associated with the patient). Based on whether the forecast satisfies the control-limit threshold(s), if the forecast is within acceptable control limits, then method 200 may end or may continue to step 275 and then step 280, in which case, additional measurements or observations of the urinary variables are received from the patient. As described above, the additional measurements or observations are added to the measurement set time series and may be used to generate an updated forecast. However, if the forecast falls outside of the control-limit threshold(s), then method 200 proceeds to step 270 and one or more actions may be invoked. For example, the decision support tool may emit an alert to a caregiver via a decision support application 140, display a warning on a graphical user interface (such as user/clinician interface 142), generate a recommendation regarding the patient's disposition or care, or other action as described herein. In an embodiment, the decision support tool further determines whether the patient requires intensified monitoring or intervention to prevent or manage recurrent urolithiasis, and may provide specific recommendations or care or may automatically schedule intervention by caregivers, consultations by specific caregivers, other healthcare resources (such as diagnostics or orders), or additional or modified care. In some embodiments, an application and graphical user interface are provided for displaying information related to the one or more actions and/or displaying aspects of the forecast generated for the target patient at step 260.

### EXAMPLE REDUCTION TO PRACTICE

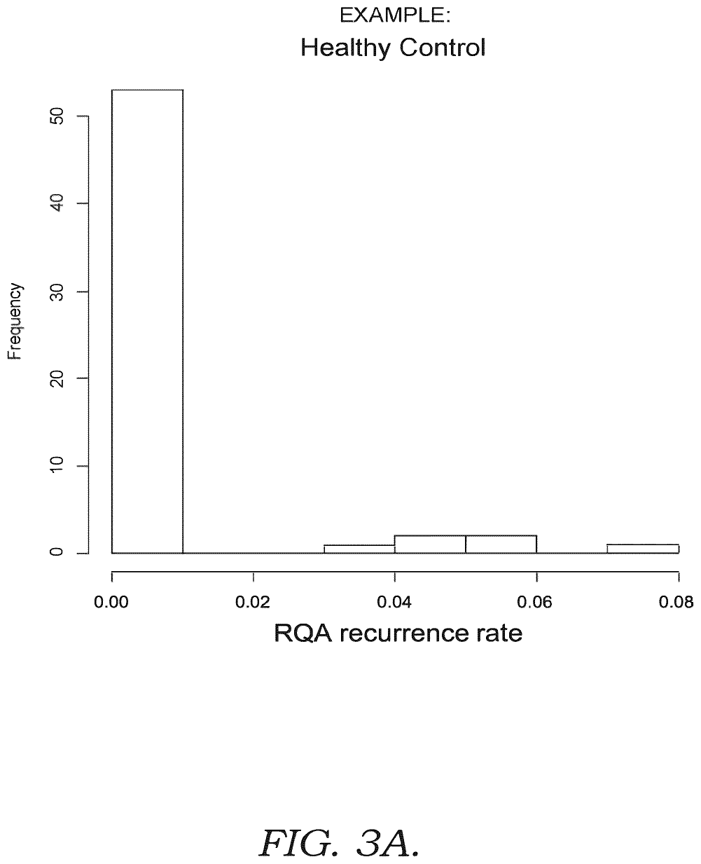

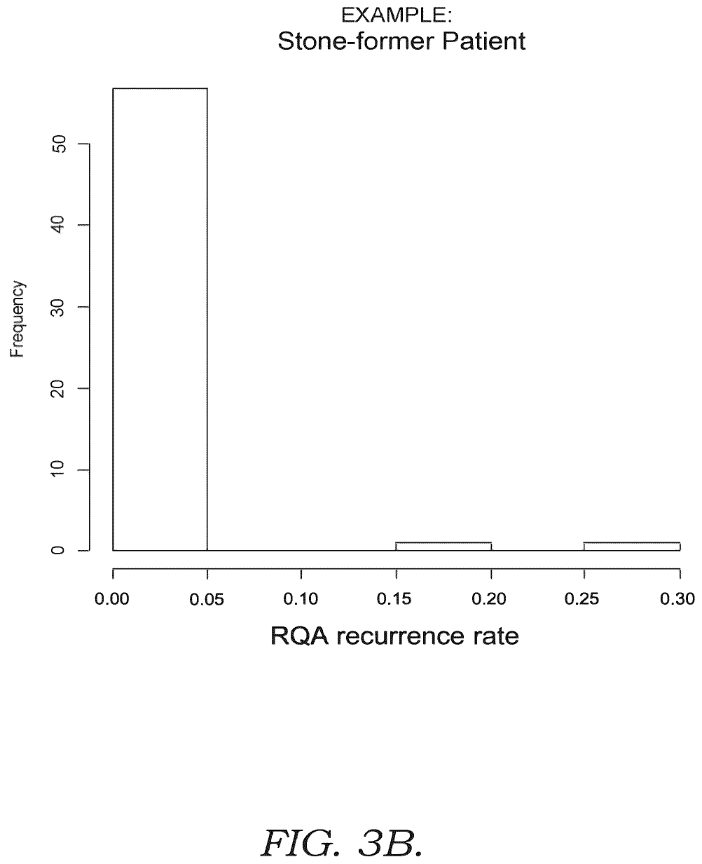

With reference now to FIGS. 3A-6 and continuing reference to FIGS. 1A and 2, an example embodiment actually reduced to practice is described. This example reduced to practice used a server cluster (computer system 120) running the Linux operating system (operating system 129), the open-source statistical software package R, and the R modules nonlinearTseries, psd, and wmtsa (computation services 126). A set of de-identified, secondary-use-consented, EHR-derived, HIPAA-compliant urinalysis and clinical chemistry measurements from 23 human patients whose 2 or more symptomatic urolithiasis episodes had previously been completed and for whom recurrence outcomes were already known was extracted from a commercially-available datawarehouse (Cerner Health Facts®). Initiating monitoring and analysis upon the first recurrence (second occurrence) of stone promotes cost-effectiveness of this embodiment and, in the instance of the prospective validation study, insured that the likely recurrence event-rate would be sufficiently high as to facilitate detection of a statistically significant result with a small cohort and relatively short follow-up time interval.

An Atago Model 3749 hand-held pen digital refractometer with 4-digit precision for urine specific gravity measurements (e.g., measurement device 141) was utilized for this example embodiment. Consented subjects were instructed to follow their usual daily routine as regards food and fluids intake and were instructed to perform measurements once per day soon after rising in the morning. The measurements entailed urinating in a small plastic specimen cup, followed by immediate dip testing and recording of test results. The specimens were then discarded. Specific gravity values were transformed into estimates of urine osmolality via a conventional linear regression model.

Consecutive values of the osmolality estimates were assembled into time series for the further processing, such as described in method 200. Urinalysis time-series Hölder exponents and RQA recurrence rates were computed for each of the 23 subjects and next-value forecasts were generated for 60-day monitoring time series. FIG. 3A and 3B show an example of RQA recurrence rates for a healthy control and an example actual recurrent stone-former patient's urine specific gravity time series over the course of one year, respectively. A logistic regression model was created utilizing the largest Hölder exponent and the RQA recurrence rate as independent (predictor) variables for a prediction time horizon of 3 years, with recurrence outcome ascertainment from PCP clinician EHR records.

Long-term trends in urinalysis analytes were, in general, absent in the 3-year follow-up data acquired from the study cohort. Spectrum analysis of long time series showed peaks corresponding to diurnal patterns of kidney function during waking and sleeping hours (e.g., more concentrated urine in the morning, following no water intake during sleep hours) but no spectral features that were predictive of 3-year likelihood of symptomatic recurrent stones.

In slightly varied embodiment, additional serial measurements of urinary calcium concentration, phosphate concentration, and chloride concentration were performed using a handheld digital spectrophotometer with a built-in 4 mL cuvette and plastic reagent strips. While results were available within 120 seconds, the measurement method was found to be too complex for most patients and cost several dollars in reagent strip expendables for each measurement. Furthermore, none of the results from the measurements reached levels of statistical significance in predicting multi-year likelihood of stone recurrence.

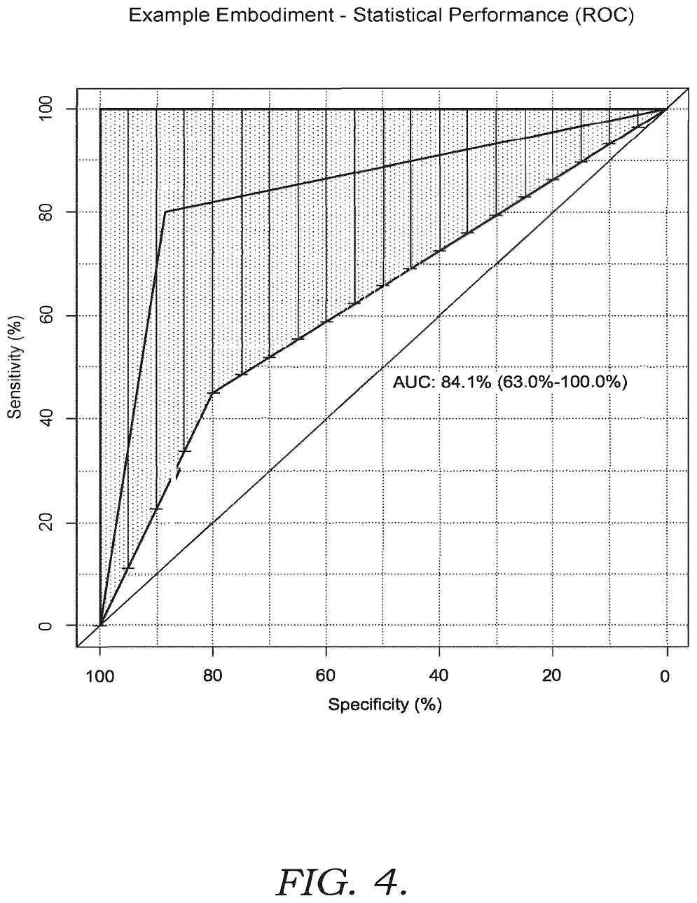

FIG. 4 shows the statistical performance from testing the example embodiment actually reduced to practice, including a receiver operating characteristic (ROC) curve as applied to the computation of the exemplary 3-year forecasts for symptomatic urolithiasis recurrence. As is known to those practiced in the art, the area under the ROC curve is a standard means of quantitatively assessing a classifier model's discrimination, the degree to which the model is able to accurately categorize cases into one or the other of two classes or categories—in this instance, “will experience symptomatic urolithiasis within 3 years” vs. “no symptomatic stones within 3 years.” Thus as can be seen, this particular example reduced to practice performs satisfactorily and represents an improvement over conventional technologies or approaches to treating similar patients. The statistical sensitivity, specificity, and ROC area-under the curve (AUC) of the logistic regression model were computed using the computer program routine illustratively depicted in FIG. 6.

Many different arrangements of the various components depicted, as well as components not shown, are possible without departing from the spirit and scope of the present disclosure. Embodiments of the technology disclosed herein have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

For example, some embodiments may include:

Embodiment 1: A decision support tool for treating a human patient prone to urolithiasis. The decision support tool comprising: a computer processor; computer memory storing computer-readable instructions that when executed by the computer processor perform operations comprising: receiving a plurality of measurements of one or more urinary variables for the patient; determining a time series of measurements from the received plurality of measurements; based on the time series of measurements, determining a set of Hölder exponents and an RQA recurrence rate; utilizing the RQA recurrence rate and at least a subset of the set of Hölder exponents in a predictive model; utilizing the predictive model, generating a forecast of the likelihood of urolithiasis for the patient over a future time interval; and based on the generated forecast, determining to initiate an intervening action.

Embodiment 2: Embodiment 1, wherein the intervention action comprises one or more of modifying treatment of the patient, ordering additional diagnostics for the patient, scheduling treatment or diagnostics for the patient, and issuing a notification to a caregiver associated with the patient.

Embodiment 3: Any of embodiments 1-2, wherein the predictive model comprises a logistic regression model.

Embodiment 4: Any of embodiments 1-3, wherein the plurality of measurements comprises a sequence of measurements, each subsequent measurement obtained after at least a minimum time interval has elapsed from the previous measurement.

Embodiment 5: Any of embodiments 1-4, wherein the minimum time interval is 4 hours.

Embodiment 6: Any of embodiments 1-5, wherein the one or more urinary variables comprise urine osmolality.

Embodiment 7: Any of embodiments 1-6, wherein the urine osmolality is determined based on an estimation from a measured specific gravity of the urine.

Embodiment 8: Any of embodiments 1-7, wherein the future time interval comprises 3 years.

Embodiment 9: Any of embodiments 1-8, wherein the time series comprises at least 60 measurements.

Embodiment 10: A computer-performed method of periodically monitoring at least one patient. The method comprising: a) collecting serial quantitative measurements of at least one physicochemical property of the urine of a patient from one or a plurality of inputs; b) periodically computing a forecast for the likelihood of forming symptomatic stones in the urinary tract, based on the time series of successive measurements; c) communicating the risk prediction or forecast messages via electronic means; and d) using the computed prediction to monitor the patient and make decisions about the need for intensified monitoring or intervention to prevent or manage recurrent urolithiasis.

Embodiment 11: A computer-performed method for assessing a human patient. The method comprising: a) receiving date-time stamped medical data about the patient from one or more data sources, wherein the medical data comprises data points from a plurality of times; b) computing from a plurality of at least 90 time points the patient's predicted risk of symptomatic urolithiasis recurrence based on the selected data from those time points; c) forecasting into the future with a time horizon during which the likelihood of recurrence of symptomatic urolithiasis is estimated; and d) emitting a report or electronic message to a human decision-maker regarding said predicted risk.

Embodiment 12: Embodiment 11, wherein numerical specific gravity and, optionally, other urinalysis data are acquired for computing the osmolality, storing a plurality of serial determinations thereof, and processing such time series to forecast the numerical value of the urolithiasis risk score at one or a plurality of future time points.

Embodiment 13: Any of embodiments 11-12, wherein the processing comprises calculations of the Hölder exponent and the recurrence quantification analysis (RQA) recurrence rate of said time series.

Embodiment 14: Any of embodiments 11-13, further comprising combining the numeric values by thresholding, Boolean logical operations, or weighted-sum.

Embodiment 15: Any of embodiments 11-14, wherein the processing includes determining an arithmetic mean or a median of the combined values.

Embodiment 16: Any of embodiments 11-15, wherein the combining uses exponentially weighted moving average or other linear combination of combined values.

Embodiment 17: Any of embodiments 11-16, wherein the combining uses the minimum or the maximum of the combined values.

Embodiment 18: Any of embodiments 11-17, wherein the combining applies a digital filter to the combined values.

Embodiment 19: Any of embodiments 11-18, wherein the processing includes determining a probability of stone recurrence from a joint relationship of the Hölder exponent and the recurrence quantification analysis (RQA) recurrence rate of the time series of urine specific gravity or osmolality.

Embodiment 20: Any of embodiments 11-19, wherein a logical conjunction of the largest Hölder exponent's exceeding 0.1 and the RQA recurrence rate's exceeding 0.2 denotes significant risk of recurrent urolithiasis meriting reporting, and electronic communication and clinical attention and action as indicated.

Embodiment 21: Any of embodiments 11-20, wherein a state of lithogenicity is determined to be related to calcific stone formation.

Embodiment 22: Any of embodiments 11-21, wherein a state of lithogenicity is determined to be related to uric acid stone formation.

Embodiment 23: Any of embodiments 11-22, wherein a state of lithogenicity is determined to be related to stones of mixed mineral-organic composition.

Embodiment 24: Any of embodiments 11-23, wherein a state of lithogenicity is determined to be related to one or more of hypocitraturia, oliguria or dehydration, spinal cord injury or other musculoskeletal or neurological condition, chronic or recurrent infection, chronic kidney disease, exposure to chemical or biological agents, an inflammatory condition or autoimmune condition, an administration of a systemically or regionally or locally applied pharmaceutical or other therapy, and hyperparathyroidism or other metabolic bone disease.

Embodiment 25: Any of embodiments 11-24, wherein measurements are performed with a frequency between 1 and 6 times per 24 hours, preferably between 1 and 2 times per 24hours.

Embodiment 26: Any of embodiments 11-25, wherein the report or electronic message may optionally include a graphical display or time-oriented plot of the trend of multiple serial determinations of the risk.

Embodiment 27: Any of embodiments 11-26, wherein the forecasting time horizon is at least one year into the future, preferably between 2 years and 10 years, and more preferably between 3 and 5 years.

Embodiment 28: Any of embodiments 11-27, wherein serial monitoring and risk calculations, forecasting, or trend analysis is initiated upon the first recurrence (second occurrence) of symptomatic urinary tract stone.

Embodiment 29: Any of embodiments 11-28, wherein specific gravity is determined by optical refractometry, by either analog or digital means, and either by specimen aliquot application to the optical surface or by immersion of the specific gravity probe optical surface into the bulk liquid specimen.

Embodiment 30: Any of embodiments 11-29, wherein a same type of specific gravity measurement apparatus is used for all measurements, not intermixing measurements from different types of refractometers.

Embodiment 31: Any of embodiments 11-30, wherein a specific gravity refractometry apparatus is used to determine the specific gravity and wherein the apparatus is subjected to periodic calibration to insure its measurement accuracy across the range of specific gravity from 1.000 to 1.040.

Embodiment 32: Any of embodiments 11-31, wherein a probe-type immersion specific gravity apparatus is permitted to thermally equilibrate in the liquid for at least 10 sec prior to making the specific gravity measurement.

Embodiment 33: Any of embodiments 11-32, wherein the osmolality or specific gravity measurement apparatus is provisioned with temperature-compensating means.

Embodiment 34: Any of embodiments 11-33, wherein specific gravity is consistently measured at approximately the same temperature each time, either immediately after collection when the specimen is still near body core temperature (37±3° C.) or after cooling to room temperature (22±3° C.).

Embodiment 35: Any of embodiments 11-34, wherein the specific gravity measurement apparatus possesses at least 3-digit accuracy and precision.

Embodiment 36: Any of embodiments 11-35, wherein a measurement from specific gravity measurement apparatus having more than 3-digit resolution is rounded to the nearest 3-digit value.

Embodiment 37: Any of embodiments 11-36, wherein each specimen is at least 20 mL of liquid and is well-mixed and homogeneous.

Embodiment 38: Any of embodiments 11-37, wherein each specimen is measured within 120 min from the time of collection.

Embodiment 39: Any of embodiments 11-38, wherein each specimen not measured immediately is kept in a tightly-covered container until measurement is performed, so as to prevent evaporation or contamination.

Embodiment 40: Any of embodiments 11-39, wherein the clean urine specimen is obtained mid-stream, after allowing 20 mL or more of initial urine output to be discarded.

Embodiment 41: Any of embodiments 11-40, wherein specific gravity values may be optionally substituted by directly-measured osmolality values, the osmolality measuring apparatus having at least 3-digit precision and accuracy in the range between 100 mOsm and 1200 mOsm.

Embodiment 42: Any of embodiments 11-41, wherein measured specific gravity values are converted into estimated osmolality values by a regression equation such as is known to those practiced in the art.

Embodiment 43: Any of embodiments 11-42, wherein occasional specimen collections may be missed or omitted but the frequency of such omissions comprises not more than 10% of the anticipated collections that would accrue in any given month.

Embodiment 44: Any of embodiments 11-43, wherein collections include a specimen from first voiding after daily sleep (usually soon after the patient awakens in the morning).

Embodiment 45: Any of embodiments 11-44, wherein one or more other collections are obtained during the course of the day whose collection timing is aperiodic and ad lib according to the individual's convenience and need to urinate, which naturally varies by food and liquid intake and ambient temperature and exertion and other factors.

It will be understood that certain features and subcombinations are of utility and may be employed without reference to other features and subcombinations and are contemplated within the scope of the claims. Not all steps listed in the various figures need be carried out in the specific order described.

What is claimed is:

1. A decision support tool for treating urolithiasis, comprising:
a computer processor;
computer memory storing computer-readable instructions that when executed by the computer processor perform operations comprising:
receiving a plurality of measurements of one or more urinary parameters;
determining a time series of measurements from the received plurality of measurements;
based on the time series of measurements, determining a set of Hölder exponents and an RQA recurrence rate;
utilizing the RQA recurrence rate and at least a subset of the set of Hölder exponents in a mathematical predictor;
utilizing the mathematical predictor, generating a forecast of the likelihood of urolithiasis over a future time interval; and
based on the generated forecast, initiating an intervening action.
